(19) United States
(12) Patent Application Publication
(10) Pub. No.: US 2023/0030901 A1
(43) Pub. Date: Feb. 2, 2023

(54) MEDICAL COIL, METHOD FOR MANUFACTURING THE SAME, AND MEDICAL DEVICE

(71) Applicant: OLYMPUS CORPORATION, Tokyo (JP)

(72) Inventor: Ryuichi SASE, Hachioji-shi (JP)

(73) Assignee: OLYMPUS CORPORATION, Tokyo (JP)

(21) Appl. No.: 17/957,126

(22) Filed: Sep. 30, 2022

Related U.S. Application Data
(63) Continuation of application No. PCT/JP2020/020000, filed on May 20, 2020.

(51) Int. Cl. A61B 17/29

(52) U.S. Cl. CPC A61B 17/29; A61B 2017/0034

(57) ABSTRACT

A medical coil includes a multi-layer coil including a plurality of winding layers in which metal strands are spirally wound, the multi-layer coil including: an inner layer in the multi-layer coil made up of one or more metal round wires; and an outer layer of the multi-layer coil made up of one or more metal flat wires. The inner layer and the outer layer are contact with each other.

### TECHNICAL FIELD

The present disclosure relates to a medical coil, a method for manufacturing the same, and a medical device. This application is a continuation application based on PCT Patent Application No. PCT/JP2020/020000, filed May 20, 2020, the content of which is incorporated herein by reference.

### BACKGROUND

The medical coil is used, for example, in a medical device such as a treatment tool that is inserted through a treatment tool channel of an endoscope.

The medical coil is inserted through a bent treatment tool channel. When changing an orientation of a treatment portion provided at a distal end or the device, the medical coil is rotated inside the bent treatment tool channel.

The medical coil is required to have “rotational transmissibility” that is an ability to transmit rotation input at a hand side to the distal end, and “compressive resistance” that is an ability to maintain a shape against a compressive force.

For example, Japanese Unexamined Patent Application, First Publication No. 2007-260248 proposes a flexible sheath in which a densely wound round wire coil having excellent rotational transmissibility is disposed outside a coarsely wound flat wire coil having excellent compressive resistance.

### SUMMARY

According to a first aspect of a medical coil having a plurality of winding layers in which metal strands are spirally wound, and the plurality of winding layers form a multi-layer coil, the medical coil includes an inner layer in the multi-layer coil which is made up of one or more metal round wires; and an outer layer of the multi-layer coil which is made up of one or more metal flat wires.

A method for manufacturing a medical coil according to a second aspect has a first process of winding one or more metal round wires around a core metal to form at least one layer of round wire coil, and a second process of winding one or more metal flat wires around an outermost periphery of the round wire coil to form a flat wire coil. A medical device according to a third aspect includes the medical coil of the first aspect.

### BRIEF DESCRIPTION OF THE DRAWINGS

FIG. 1 is a schematic front view showing a configuration example of a medical device according to a first embodiment of the present disclosure.

FIG. 2 is a cross-sectional view taken along a line A-A of FIG. 1.

FIG. 3 is a schematic cross-sectional view showing a configuration example of a medical coil according to the first embodiment of the present disclosure.

FIG. 4 is a schematic diagram showing an example of a first process of a method for manufacturing a medical coil according to the first embodiment of the present disclosure.

FIG. 5 is a schematic diagram showing an example of a second process of the method for manufacturing the medical coil according to the first embodiment of the present disclosure.

FIG. 6 is an operation explanatory diagram of the medical coil according to the first embodiment of the present disclosure.

FIG. 7 is a schematic cross-sectional view showing an example of a medical coil of a modified example.

FIG. 8 is a diagram for explaining an operation of the medical coil of the modified example.

FIG. 9 is a schematic cross-sectional view showing a configuration example of a medical coil according to a second embodiment of the present disclosure.

FIG. 10 is a schematic diagram showing a rotational transmissibility test device.

FIG. 11 is a schematic diagram showing an example of a compressive resistance test device.

### DETAILED DESCRIPTION OF THE PREFERRED EMBODLMENTS

Hereinafter, each embodiment of the present disclosure will be described with reference to the attached drawings. In all the drawings, even if the embodiments are different, the same or corresponding members are denoted by the same reference numerals, and common description will be omitted.

In the present specification, there is a case in which a preferable numerical range may be exemplified, for example, as “X (lower limit value) or more, Y (upper limit value) or less”, or the like in regard to dimensional values. When a plurality of preferable numerical ranges are exemplified in regard to the dimensional values, a numerical range in which combination of a lower limit value and an upper limit value is appropriately changed within the widest numerical range is also a preferable range unless otherwise specified.

### First Embodiment

The medical coil and the medical device of the first embodiment will be described.

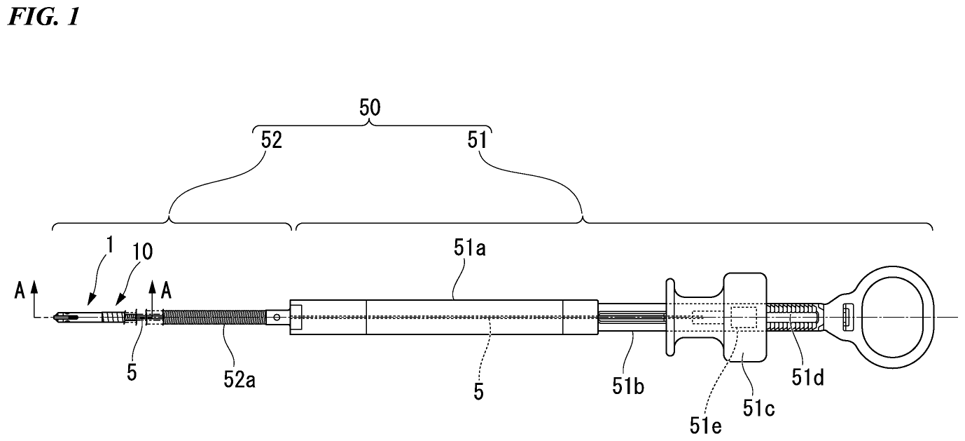

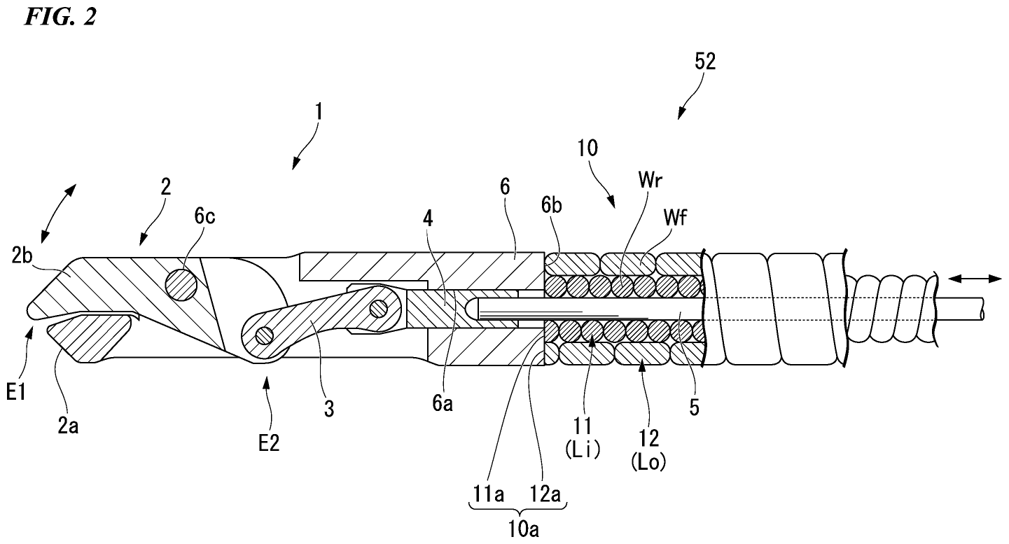

FIG. 1 is a schematic front view showing a configuration example of a medical device according to the first embodiment of the present disclosure. FIG. 2 is a cross-sectional view taken along the line A-A of FIG. 1.

A grasping forceps 50 (medical device) shown in FIG. 1 is an example of the medical device of the first embodiment. The grasping forceps 50 is configured to be inserted into the patient's body through an endoscopic treatment tool channel.

The grasping forceps 50 has a manipulation portion 51 and an insertion portion 52 in this order from a proximal end to a distal end in the insertion direction. In the grasping forceps 50, a long manipulation shaft 5 is inserted into the manipulation portion 51 and the insertion portion 52.

The manipulation shaft 5 transmits the linear motion of the manipulation portion 51 to the distal end of the insertion portion 52. For example, the manipulation shaft 5 is a metal stranded wire or a single metal wire made of a material such as SUS304. A diameter of the manipulation shaft 5 may be 0.1 mm or more and 1.4 mm or less, and more preferably 0.3 mm or more and 0.8 mm or less, for example.

The manipulation portion 51 is disposed outside the endoscope and is manipulated by an operator.

The manipulation portion 51 has a cylinder 51a, a guide member 51b, and a slider 51c.

The cylinder 51a is formed in a cylindrical shape. The manipulation shaft 5 is inserted into the cylinder 51a.

A guide member 51b extends in an axial direction of the cylinder 51a at the proximal end (right side in FIG. 1) of the cylinder 51a.

The guide member 51b has a columnar shape so as to slidably support the slider 51c. A guide hole 51d extending in a longitudinal direction of the slider 51c penetrates a central portion of the slider 51c in a radial direction. The slider 51c is slidably fitted into the guide hole 51d.

A proximal end of the manipulation shaft 5 inserted into the cylinder 51a and the guide hole 51d is fixed to the slider 51c. The slider 51c pulls the manipulation shaft 5 toward a proximal side by moving toward the proximal side along the guide member 51b.

The slider 51c is provided with a stopper 51e that can be brought into contact with and separated from the guide member 51b. The stopper 51e fixes the slide position of the slider 51c when it comes into contact with the guide member 51b.

The insertion portion 52 is a long member that is inserted into the treatment tool channel of the endoscope. The insertion portion 52 has an enough length to be inserted into the patient's body through the opening at the distal end of the treatment tool channel.

The insertion portion 52 has a forceps portion 1, a coil sheath 10 (medical coil), and a proximal end sheath 52a in this order from the distal end toward the proximal end. The coil sheath 10 is an example of the medical coil of the present embodiment.

As shown in FIG. 2, the forceps portion 1 has a main body 6, a grasper 2, a slider 4, and a link 3.

The main body 6 is made of a columnar hard member.

A first grip 2a forming a part of the grasper 2 to be described later is provided at a distal end of the main body 6.

A guide hole 6a is formed axially in the central portion of the proximal end 6b of the main body 6. The guide hole 6a slidably supports the slider 4, which will be described later.

A distal end 10a of the coil sheath 10, which will be described later, is connected to an outer peripheral portion of the proximal end 6b of the main body 6.

The grasper 2 grasps, for example, a living tissue. The grasper 2 has a first grip 2a fixed to the distal end of the main body 6, and a second grip 2b provided to be brought into contact with and separated from the first grip 2a.

The second grip 2b is a lever that is pivotably fixed to a pivot support shaft 6c disposed near the first grip 2a in the main body 6.

A first end portion E1 of the second grip 2b is configured to be brought into contact with and separated from the first grip 2a according to the rotation position.

A distal end of the link 3 is connected to a second end portion E2 on the opposite side of the first end portion E1 with the pivot support shaft 6c interposed therebetween.

A proximal end of the link 3 is connected to a distal end of the slider 4, which will be described later, and converts a linear motion of the slider 4 into a pivoting of the second grip 2b.

The slider 4 is a shaft member that can slide along the longitudinal direction of the guide hole 6a of the main body 6. The proximal end of the link 3 is connected to the distal end of the slider 4. The distal end of the manipulation shaft 5 is connected to a proximal end of the slider 4.

The coil sheath 10 of the present embodiment includes a multi-layer coil including a round wire coil 11 and a flat wire coil 12. The round wire coil 11 and the flat wire coil 12 form a plurality of winding layers in which a metal strand in the present embodiment is spirally wound.

The round wire coil 11 is formed by spirally winding a metal round wire Wr, which is a metal strand.

The metal round wire Wr is made of a metal such as stainless steel, and is a single wire having a substantially circular cross-sectional shape (hereinafter, simply referred to as a cross-sectional shape) that is orthogonal to the longitudinal direction.

The material of the metal round wire Wr is, for example, SUS304, SUS316, SUS631J1, and SUS301, or the like.

The cross-sectional shape of the metal round wire Wr used for the round wire coil 11 is more preferably a perfect circle, but it may not be a strict perfect circle. For example, the cross-sectional shape of the metal round wire Wr may be an approximate shape of a circle, an ellipse, an approximate shape of an ellipse, or the like. When the cross-sectional shape of the metal round wire Wr is a non-round shape, it may be a non-round shape caused by a manufacturing error, or it may be a non-circular shape that is intentionally processed.

When a size of a maximum diameter in the cross-sectional shape of the metal round wire Wr is defined as B and a diameter in the direction orthogonal to the maximum diameter is defined as A, a ratio B/A represents the degree of non-roundness in the cross-sectional shape.

The ratio B/A may be 1 or more and 1.1 or less, and more preferably 1 or more and 1.05 or less.

Hereinafter, unless otherwise specified, the diameter of the metal round wire Wr means an average diameter.

The diameter of the metal round wire Wr is preferably small from the viewpoint of reducing the diameter of the coil sheath 10 and improving the flexibility. The diameter of the metal round wire Wr is preferably large to increase the rigidity from the viewpoint of maintaining good rotational transmissibility and compressive resistance.

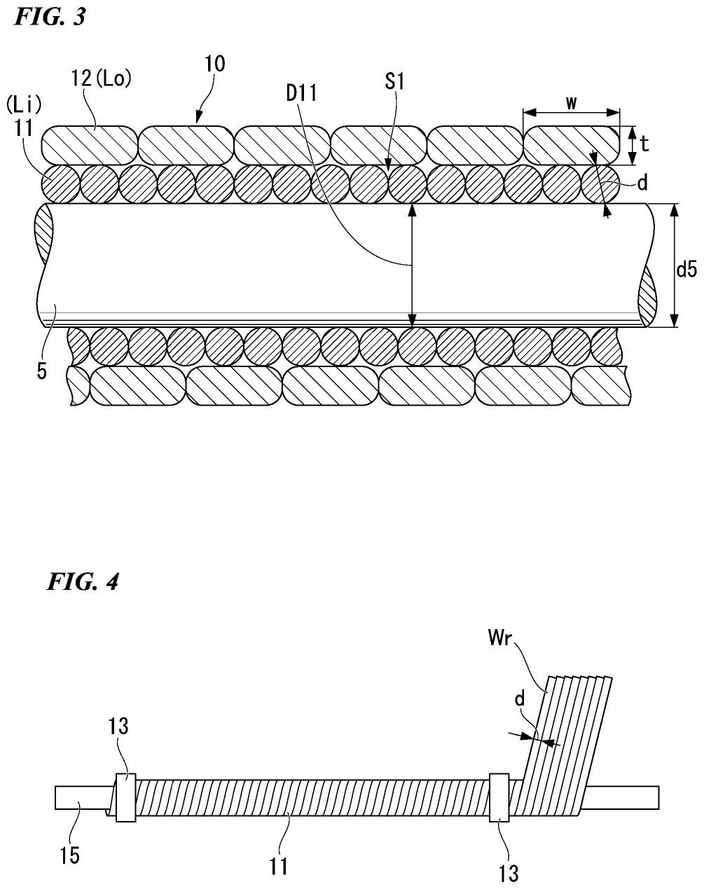

FIG. 3 is a schematic cross-sectional view showing a configuration example of a medical coil according to the first embodiment of the present disclosure.

For example, when the diameter of the metal round wire Wr is defined as d, d may be 0.1 mm or more and 0.3 mm or less, and more preferably 0.1 mm or more and 0.2 mm or less.

Since FIG. 3 is an axial cross section including the central axis of the manipulation shaft 5, strictly speaking, the cross section shown in FIG. 3 is not a cross section orthogonal to an extending direction (a winding direction) of each metal round wire Wr. For example, when the metal round wire Wr is a perfect circle, although the shape of the axial cross section of the round wire coil 11 is an ellipse, it is schematically drawn as a circle in FIG. 3. The diameter d of the metal round wire Wr shown in the drawing indicates a dimensional value of a cross section orthogonal to the extending direction of the metal round wire Wr. The cross section of the flat wire coil 12 is also similarly modeled, and w and t also indicate dimensional values of the cross section orthogonal to the extending direction (the winding direction) of the metal flat wire Wf, similarly to the diameter d.

The round wire coil 11 is densely wound.

When the round wire coil 11 is “densely wound”, it means that the metal round wires Wr adjacent to each other in the axial direction of the round wire coil 11 are in close contact with each other, or the distance between the surfaces of the metal round wires Wr adjacent to each other is 10% or less of the diameter of the metal round wire Wr.

The size of the inner diameter of the round wire coil 11 in the coil sheath 10 is substantially equal to the diameter d5 of the manipulation shaft 5 so that the manipulation shaft 5 can be inserted. The manipulation shaft 5 can smoothly move in the axial direction inside the round wire coil 11.

For example, when the inner diameter of the round wire coil 11 is defined as D11, (D11-d5) may be 0.01 mm or more and 0.4 mm or less, and more preferably 0.05 mm or more and 0.3 mm or less.

The round wire coil 11 is formed by winding N metal round wires Wr (N is a natural number) in N rows. For example, N may be 1 or more and 16 or less, and more preferably 4 or more and 8 or less. The round wire coil 11 may be formed by winding one or more metal round wires Wr.

The winding direction of the round wire coil 11 may be S winding or Z winding. In the example shown in FIG. 2, the winding is S winding. The S winding is a winding method in which the inclination of the spiral descends to the right when the axial direction of the coil is aligned in the vertical direction and viewed in the radial direction from the outside. The S winding can be said to be a winding method along striation of the left-handed screw.

The Z winding is a winding method in which the inclination of the spiral ascends to the right when the axial direction of the coil is aligned in the vertical direction and the coil is viewed in the radial direction from the outside. The Z winding can be said to be a winding method along the striation of a right-handed screw.

The flat wire coil 12 is formed by spirally winding a metal flat wire Wf, which is a metal strand, around an outer peripheral portion of the round wire coil 11.

The metal flat wire Wf is made of, for example, a metal such as stainless steel. The metal flat wire Wf is a single wire having an aspect ratio of more than 1 in a cross-sectional shape (hereinafter, simply cross-sectional shape) orthogonal to the longitudinal direction. When the long direction and the short direction orthogonal to each other are specified in the cross-sectional shape, and the maximum length in the long direction is defined as b, and the maximum length in a short direction is defined as a, the aspect ratio of the metal flat wire Wf is defined by a ratio of b/a. The long direction and the short direction for obtaining the aspect ratio are specified so that the distance between a plane extending in the long direction and a plane extending in the short direction of the cross-sectional shape of the metal flat wire Wf is minimized as a whole.

The cross-sectional shape of the metal flat wire Wf may be, for example, a rectangle, a rectangle with rounded four corners, an ellipse, an oval with both end portions rounded into a semicircle, and similar shapes thereof.

For example, the cross-sectional shape of the metal flat wire Wf in the flat wire coil 12 shown in FIG. 3 is a rectangle with rounded four corners. hi this case, the long direction of the cross-sectional shape of the metal flat wire Wf is a long side direction, and a maximum length of the long direction is a width w in the long side direction. Similarly, the short direction is a short side direction, and a thickness tin the maximum long short side direction.

For example, when the cross-sectional shape of the metal flat wire Wf is elliptical, the long direction is a major axis direction, and the maximum length is a major axis of the ellipse. Similarly, the short direction is the minor axis direction, and the maximum length in the short direction is a minor axis.

Hereinafter, the maximum length in the long direction of the metal flat wire Wf is referred to as a width, and the maximum length in the short direction is referred to as a thickness, regardless of the cross-sectional shape.

For example, the aspect ratio of the metal flat wire Wf may be more than 1 and 3 or less, and more preferably 1.5 or more and 2.5 or less.

In the present embodiment, the aspect ratio of the metal flat wire Wf used for the flat wire coil 12 is larger than the ratio B/A of the metal round wire Wr used for the round wire coil 11.

The width w of the metal flat wire Wf used for the flat wire coil 12 may be 1 time or more and 3 times or less the diameter d of the metal round wire Wr, and more preferably, 1 time or more and 2 times or less.

When w/d is less than 1, the metal flat wire Wf easily enters a V-shaped gap S1 formed on the surfaces of the metal round wires Wr adjacent to each other in the axial direction of the coil sheath 10.

If w/d exceeds 3, the coil sheath 10 is less likely to bend, and the rotational transmissibility decrease.

For example, w may be 0.1 mm or more and 0.7 mm or less, and more preferably 0.3 mm or more and 0.5 mm or less.

The thickness t of the metal flat wire Wf used for the flat wire coil 12 may be 0.5 times or more and 3 times or less the diameter d of the metal round wire Wr, and more preferably 0.8 times or more and 1.5 times or less.

If t/d is less than 0.5, because the rigidity of the flat wire coil 12 in the radial direction decreases, the compressive resistance performance in the radial direction decrease.

If t/d exceeds 3, the outer diameter of the coil sheath 10 may become too large or the flexibility of the coil sheath 10 may decrease too much.

For example, t may be 0.1 mm or more and 0.3 mm or less, and more preferably 0.1 mm or more and 0.2 mm or less.

For example, as the material of the metal flat wire Wf, the same material as the metal round wire Wr used for the round wire coil 11 can be adopted.

A method for manufacturing the metal flat wire Wf is not particularly limited. For example, the metal flat wire Wf may be manufactured by rolling the metal round wire Wr.

The flat wire coil 12 is densely wound around the outer peripheral portion of the round wire coil 11 so that the metal flat wires are adjacent to each other in the width direction of the metal flat wire. However, the flat wire coil 12 is wound around the outer peripheral portion of the round wire coil 11 to be relatively movable at least in the axial direction of the round wire coil 11.

The term that the flat wire coil 12 is “densely wound” means that the metal flat wires adjacent to each other in the axial direction of the flat wire coil 12 are in close contact with each other, or the distance between the surfaces of the metal flat wires adjacent to each other in the width direction is equal to or less than 5% of the width of the metal flat wire.

The flat wire coil 12 may be formed by winding M metal flat wires (M is a natural number) in M rows.

The flat wire coil 12 is wound to intersect the round wire coil 11 when viewed from the radial direction. As long as it intersects the round wire coil 11, the winding direction may be S winding or Z winding.

It is more preferable that the winding direction of the flat wire coil 12 is opposite to the winding direction of the round wire coil 11. In this case, as compared with a case where the winding directions are the same direction, stable rotational transmissibility can be obtained regardless of the rotation direction of the coil sheath 10.

In the example shown in FIG. 2, the flat wire coil 12 has a Z winding opposite to the S winding to correspond to the round wire coil 11 having an S winding. Hereinafter, unless otherwise specified, the winding directions of the round wire coil 11 and the flat wire coil 12 will be described as being opposite to each other.

As described above, the coil sheath 10 is a multi-layer coil in which the flat wire coil 12 is wound around the outer peripheral portion of the round wire coil 11. Therefore, the round wire coil 11 and the flat wire coil 12 are stacked concentrically. The round wire coil 11 and the flat wire coil 12 form two winding layers in which the metal strands are spirally wound.

The round wire coil 11 forms an inner layer Li in the multi-layer coil. In the present embodiment, an inner peripheral portion of the round wire coil 11 forms an inner diameter portion of the coil sheath 10.

The flat wire coil 12 forms an outer layer Lo in the multi-layer coil. In the present embodiment, an outer peripheral portion of the flat wire coil 12 forms an outer diameter portion of the coil sheath 10.

In the example shown in FIG. 3, the outer diameter of the coil sheath 10 is {D11+2×(d+t)}.

The coil sheath 10 may include a layered portion (hereinafter referred to as a non-winding layer) that is not a winding layer. In this case, the outer diameter of the coil sheath 10 may be larger than {D11+2×(d+t)} depending on the thickness of the non-winding layer.

For example, when a non-winding layer is included between the round wire coil 11 and the flat wire coil 12, the flat wire coil 12 is wound around the outer peripheral portion of the non-winding layer. In the multi-layer coil, the non-winding layer provided on the outer peripheral portion of the winding layer is defined as an outer peripheral portion of the winding layer covered with the non-winding layer.

As shown in FIG. 2, the distal end 10a of the coil sheath 10 includes a distal end 11a of the round wire coil 11 and the distal end 12a of the flat wire coil 12. In the example shown in FIG. 2, the distal ends 11a and 12a are located on the same plane orthogonal to the axial direction of the coil sheath 10. However, depending on the shape of the proximal end 6b of the main body 6, a step may be formed between the distal ends 11a and 12a.

The distal end 10a is joined to the proximal end 6b of the main body 6. A joining method is not particularly limited. For example, the distal end 10a may be joined to the proximal end 6b by brazing, soldering, caulking, welding or the like.

As shown in FIG. 1, the proximal end side of the coil sheath 10 is inserted through the proximal end sheath 52a.

The proximal end sheath 52a is fixed to the distal end of the cylinder 51a. The proximal end sheath 52a forms an insertion passage that guides the coil sheath 10 from a mouthpiece portion toward the inside of the treatment tool channel when inserted into the mouthpiece portion.

For example, the proximal end sheath 52a is a tubular member that can be inserted into the opening of the mouthpiece portion at the proximal end of the treatment tool channel. For example, the proximal end sheath 52a is made of a coil sheath that is shorter than the coil sheath 10 and harder than the coil sheath 10.

The method for manufacturing the coil sheath 10 will be described.

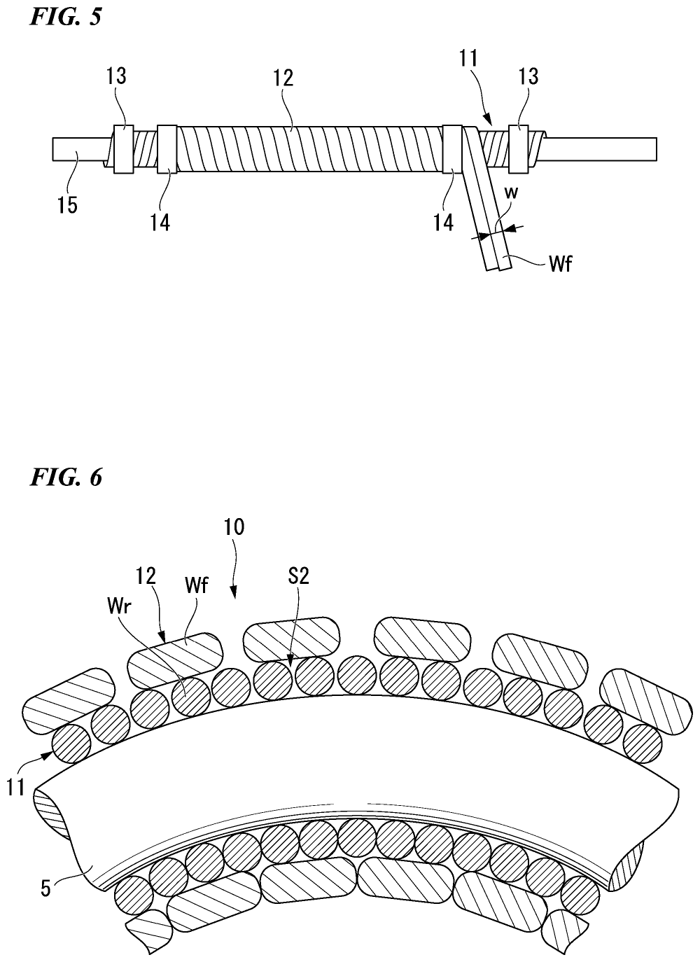

FIG. 4 is a schematic diagram showing an example of a first process of the method for manufacturing a medical coil according to the first embodiment of the present disclosure. FIG. 5 is a schematic diagram showing an example of a second process of the method for manufacturing a medical coil according to the first embodiment of the present disclosure.

The method for manufacturing the medical coil according to this embodiment includes the first process and the second process.

As shown in FIG. 4, in the first process, the metal round wire Wr is wound around a core metal 15 to form the round wire coil 11.

The metal round wire Wr is not particularly limited as long as the above-mentioned round wire coil 11 can be formed by densely winding the metal round wire Wr around the core metal 15. For example, a metal wire having a substantially circular cross-sectional shape having a diameter d is used as the metal round wire Wr.

The core metal 15 is a single wire longer than the coil sheath 10. The core metal 15 has a circular cross section having an outer diameter corresponding to the inner diameter D11 of the round wire coil 11.

The material of the core metal 15 is not particularly limited as long as a coil having an inner diameter D11 can be formed by winding the metal round wire Wr. For example, copper may be used as the material of the core metal 15.

For example, the metal round wire Wr is densely wound around the core metal 15 by a coil winding machine. As a result, the round wire coil 11 is formed on the core metal 15. The round wire coil 11 is formed to be longer than the total length of the coil sheath 10.

In the example shown in FIG. 4, eight metal round wires Wr are prepared and wound around the core metal 15 with eight threads. The winding direction of the metal round wire Wr is, for example, S winding.

Both end portions of the round wire coil 11 are fixed on the core metal 15 with crimp terminals 13. The metal round wire Wr that is not wound around the core metal 15 is cut at an appropriate portion.

The first process is ended.

In the metal round wire Wr, the metal round wires Wr adjacent to each other are brought into line contact with each other. In the metal flat wire, uneven winding may occur due to a gap in which each of the flat wire cannot contact or a surface contact. Compared to the metal flat wire, the metal round wire Wr is less likely to have uneven winding. Therefore, the metal round wire Wr is easily densely wound along the surface of the core metal 15.

An envelope surface of the outer peripheral portion of the round wire coil 11 approaches a cylindrical surface similar to a cylindrical surface of the core metal 15.

After this, the second process is performed.

As shown in FIG. 5, in the second process, the metal flat wire Wf is wound around the outer peripheral portion of the round wire coil 11 to form the flat wire coil 12.

As the type of the metal flat wire Wf, the above-mentioned appropriate type is used. For example, as the metal flat wire Wf, a metal wire having a rectangular cross-sectional shape with rounded corners having a width w and a thickness t may be used.

The metal flat wire Wf is densely wound around the outer peripheral portion of the round wire coil 11 by a coil winding machine. In the example shown in FIG. 4, two metal flat wires Wf adjacent to each other in the width direction are prepared. The two metal flat wires Wf are wound around the outer peripheral portion of the round wire coil 11 with two threads.

In the example shown in FIG. 4, the winding direction of the metal flat wire Wf is Z winding in the opposite direction corresponding to the round wire coil 11 being the S winding.

Since the metal flat wires Wf are disposed adjacent to each other in the width direction and densely wound when the flat wire coils 12 are wound around the outer peripheral portion of the round wire coil 11, the inner surface of the flat wire coil 12 becomes a cylindrical surface. Since the metal round wire Wr of the round wire coil 11 that abuts on the inner surface of the flat wire coil 12 has a substantially circular cross-sectional shape, the abutting portion with the inner surface of the flat wire coil 12 has a linear shape that extends spirally.

If the metal round wire is wound around the outer peripheral portion of the round wire coil 11, the metal round wires abut with each other in a dot shape at a position where the metal round wires intersect each other. On the other hand, in the coil sheath 10, the reaction force from the round wire coil 11 acting on the flat wire coil 12 is dispersed as compared with the case where the metal round wire is wound around the outer peripheral portion of the metal round wire coil, thereby the deformation of the flat wire coil 12 at the time of winding is suppressed. Accordingly, the flat wire coil 12 is prevented from entering the gap Si (see FIG. 3) on the surface of the round wire coil 11.

As a result, the flat wire coil 12 is smoothly wound along the cylindrical surface which is the envelope surface of the outer circumference of the round wire coil 11.

In the coil sheath 10 of the present embodiment, in addition to using the metal flat wire Wf, by making the width w of the metal flat wire Wf to be 1 times or more the diameter d of the metal round wire Wr, the metal flat wire Wf can be suppressed from entering the gap S1.

For example, if the width w of the metal flat wire Wf of the flat wire coil 12 is less than the diameter d of the metal round wire Wr, the metal flat wire Wf easily enters the gap S1 at the time of winding, although it is not as much as in the case of winding the metal round wire. If a part of the metal flat wire Wf enters the gap S1, the winding direction of the metal flat wire Wf is disturbed by the influence of the winding direction of the metal round wire Wr, or unevenness is formed on the outer peripheral surface of the flat wire coil 12.

If the winding direction of the flat wire coil 12 is disturbed, the dense winding property of the flat wire coil 12 deteriorates, thereby the flat wire coil 12 is easily compressed in the axial direction and the compressive resistance in the axial direction deteriorates.

When the dense winding property deteriorates, the portion of the round wire coil 11 that is not covered with the flat wire coil 12 increases. As a result, the resistance may be weakened against the compressive force acting in the radial direction from the outside of the coil sheath 10.

When the width w of the metal flat wire Wf is equal to or larger than the diameter d of the metal round wire Wr, the above-mentioned problems are suppressed.

When the flat wire coil 12 longer than the total length of the coil sheath 10 is formed between the crimp terminals 13, both end portions of the flat wire coil 12 are fixed on the round wire coil 11 with the crimp terminals 14.

The metal flat wire Wf that is not wound around the round wire coil 11 is cut at an appropriate site.

The second process is ended.

After that, the round wire coil 11 and the flat wire coil 12 fixed to the core metal 15 are placed in a heat treatment furnace and subjected to heat treatment so that the core metal 15 is softened.

After that, the softened core metal 15 is removed, and the round wire coil 11 and the flat wire coil 12 are cut to the length of the coil sheath 10 at both end portions.

The coil sheath 10 has been manufactured as described above.

The coil sheath 10 is incorporated into the grasping forceps 50 by fixing both end portions to the main body 6 and the cylinder 51a.

Next, the operation of the coil sheath 10 will be described.

As shown in FIG. 1, the coil sheath 10 is used for the insertion portion 52 of the grasping forceps 50. The manipulation shaft 5 is inserted into the coil sheath 10.

The insertion portion 52 is inserted into the patient's body, for example, via the treatment tool channel of the endoscope. At this time, since the insertion portion of the endoscope is bent along the body cavity, the insertion portion 52 is inserted through the bent treatment tool channel.

A surgeon moves the insertion portion 52 forward and backward inside the treatment tool channel or rotates the insertion portion 52 inside the treatment tool channel to change a grasping direction of the forceps portion 1 according to the need for the treatment. The flat wire coil 12 that forms an outermost layer of the insertion portion 52 receives an external force in the radial direction by sliding inside the treatment tool channel.

The coil sheath 10 receives a compressive force in the axial direction inside the treatment tool channel depending on the manipulation force of the manipulation portion 51, and is bent depending on the curvature of the treatment tool channel. Further, the coil sheath 10 receives the compressive force in the radial direction from the abutting portion with the treatment tool channel.

The outermost layer of the coil sheath 10 is covered with the flat wire coil 12. Since the radial force from the outside is dispersed in the width direction of the flat wire coil 12, the force is difficult to be transferred to the inner round wire coil 11. The coil sheath 10 has a compressive resistance that resists a compressive force acting in the radial direction from the outside.

The flat wire coil 12 is densely wound around the outer peripheral portion of the densely wound round wire coil 11 in a winding direction different from that of the round wire coil 11. As described in the manufacturing method, the flat wire coil 12 is smoothly wound along the envelope surface of the round wire coil 11, and unevenness on the outer peripheral surface and uneven winding are suppressed. Therefore, the advance or retreat of the treatment tool channel becomes smooth, and the compressive resistance in the axial direction is suppressed.

FIG. 6 is an operation explanatory diagram of the medical coil according to the first embodiment of the present disclosure.

As shown in FIG. 6, when the coil sheath 10 is bent, the coil sheath 10 receives a compressive force on the inside of the bend (lower side in FIG. 6) and a tensile force on the outside of the bend (upper side in FIG. 6) along the bending line.

On the outside of the bend, a winding interval of the round wire coil 11 and the flat wire coil 12 becomes wide. Since the winding interval of the flat wire coil 12 having a large radius of curvature of the bend is wider, the flat wire coil 12 moves relative to each other along the outer peripheral portion of the round wire coil 11.

At this time, because each metal flat wire Wf of the flat wire coil 12 slides on a plurality of metal round wires Wr disposed at a pitch shorter than the winding pitch of the metal flat wire Wf, the metal flat wire Wf can move smoothly. Therefore, the resistance of the coil sheath 10 when bent is low.

In particular, when the width w of the metal flat wire Wf is 1 times or more of the diameter d of the metal round wire Wr, even if the winding interval of the metal round wire Wr is opened and a gap S2 penetrating in the radial direction is formed, it is possible to prevent the metal round wire Wr from entering the gap S2. Therefore, the resistance at the time of bending can be further suppressed.

As the width w of the metal flat wire Wf increases, the number of metal round wires Wr with which the metal flat wire Wf abuts increases. As a result, the metal flat wire Wf slides smoothly on the plurality of metal round wires Wr at the time of bending, and the resistance at the time of bending is further reduced.

The coil sheath 10 is disposed in a bent treatment tool channel. For example, the coil sheath 10 may be rotated to change the grasping direction of the forceps portion 1. When the coil sheath 10 is rotated in the circumferential direction of the coil sheath 10 in a bent state, the bending direction of the coil sheath 10 changes. If the resistance to bending is large, because the amount of rotation at the bending portion decreases, the amount of rotation at the proximal end is less likely to be transferred to the distal end.

In the present embodiment, because the resistance at the time of bending can be reduced, the rotational transmissibility of the coil sheath 10 is improved.

Inside the bending of the coil sheath 10, a compressive force acts on the metal flat wires Wf adjacent to each other in the flat wire coil 12. As shown in FIG. 6, when the corners of both end portions of the flat wire coil 12 in the width direction are rounded, there is an advantage that the contact resistance between the metal flat wire Wf is reduced in the bending operation.

Here, the operation of the coil sheath 10 of the present embodiment will be described in comparison with the coil sheath of the modified example.

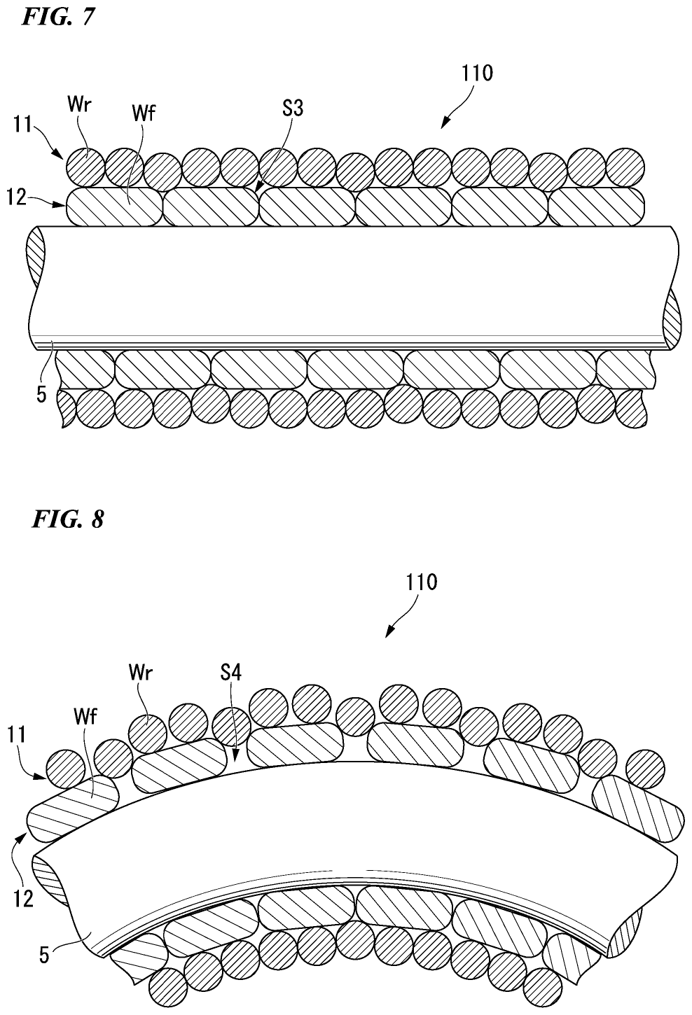

FIG. 7 is a schematic cross-sectional view showing an example of a medical coil as a modified example. FIG. 8 is a diagram for explaining an operation of the medical coil of the modified example.

As shown in FIG. 7, the coil sheath 110 of the modified example is a multi-layer coil including a round wire coil 11 and a flat wire coil 12. However, the round wire coil 11 in the coil sheath 110 forms an outer layer of the coil sheath 110, and the flat wire coil 12 forms an inner layer of the coil sheath 110.

The coil sheath 110 can be manufactured in the same manner as in the present embodiment, except that the flat wire coil 12 is wound around the core metal 15 and then the round wire coil 11 is wound around the outer peripheral portion of the flat wire coil 12.

In the example shown in FIG. 7, since the flat wire coil 12 of the coil sheath 110 is also rounded at both end portions in the width direction, a V-shaped gap S3 is formed between the roundness of the metal flat wires Wf adjacent to each other.

The round wire coil 11 easily enters the gap S3 when the round wire coil 11 is wound. Therefore, the outer diameter of the round wire coil 11 wound around the outer peripheral portion of the flat wire coil 12 varies in the axial direction, and the winding direction of the round wire coil 11 is easily disturbed. If the winding direction of the round wire coil 11 is disturbed, because a gap is generated between the round wire coils 11 adjacent to each other, the dense winding property of the round wire coil 11 deteriorates.

For example, it is conceivable to increase the diameter of the metal round wire Wr to make it difficult for the metal round wire Wr to enter the gap S3. In this case, the outer diameter of the coil sheath 110 increases.

For example, it is conceivable to make the cross-sectional shape of the metal flat wire Wf rectangular to eliminate the gap S3. In this case, since the rectangular corners interfere with each other when the coil sheath 110 is bent, the bending resistance increases.

In contrast, according to the coil sheath 10 of the present embodiment, the outer peripheral portion can be covered with a good densely wound winding layer having little change in the outer diameter, without suppressing the outer diameter and impairing the bending performance.

As shown in FIG. 8, when the coil sheath 110 is bent, the winding interval of the flat wire coil 12 on the outer side of the bending opens, and a gap S4 in which the metal flat wires Wf are separated from each other in the width direction is formed. In particular, when the gap S4 opens to be larger than the diameter of the metal round wire Wr, the metal round wire Wr easily enters the gap S4. When the metal round wire Wr enters the gap S4, because the gap S4 cannot be reduced when bending in the opposite direction, the resistance to bending increases. As the bending resistance increases, the rotational transmissibility of the coil sheath 110 decreases.

In contrast, since the metal flat wire Wf is wound on the outside in the coil sheath 10 of the present embodiment, even if the gap S2 of the inner round wire coil 11 is formed as much as the gap S4 at the time of bending, the flat wire coil 12 is hard to enter the gap S2 as compared with the case where the metal round wire Wr is wound as in the modified example. As a result, the coil sheath 10 can be smoothly bent, and good rotational transmissibility can be obtained.

As described above, according to the coil sheath 10 of the present embodiment, the rotational transmissibility is excellent even if the outer diameter is small. According to the grasping forceps 50 of the present embodiment, since the forceps portion 1 is provided at the distal end of the coil sheath 10, the diameter of the insertion portion 52 can be reduced and maneuverability of the forceps portion 1 can be improved.

### Second Embodiment

A medical coil of the second embodiment will be described.

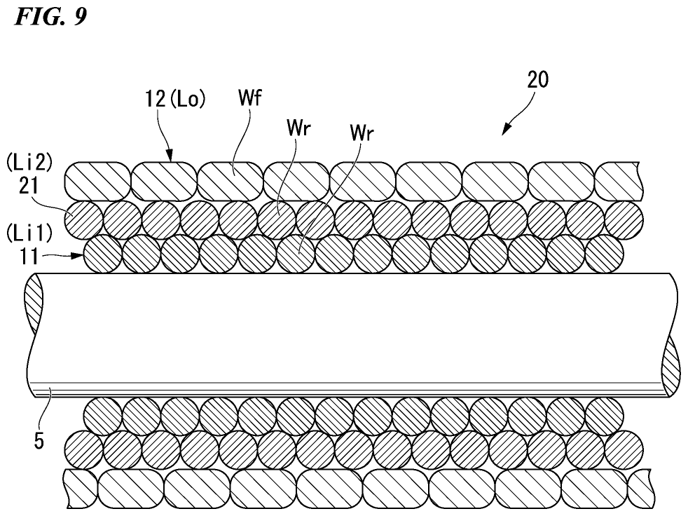

FIG. 9 is a schematic cross-sectional view showing a configuration example of the medical coil according to the second embodiment of the present disclosure.

As shown in FIG. 9, in a coil sheath 20 (medical coil) of the present embodiment, a round wire coil 21 is added to the coil sheath 10 of the first embodiment. The coil sheath 20 can be used in place of the coil sheath 10 in the grasping forceps 50 of the first embodiment.

Hereinafter, the points different from the first embodiment will be mainly described.

The round wire coil 21 is formed by spirally winding the metal round wire Wr around the outer peripheral portion of the round wire coil 11. However, the winding direction of the round wire coil 21 is different from the winding direction of the round wire coil 11. For example, when the round wire coil 11 is Z winding, the round wire coil 21 is S winding.

The round wire coil 21 is densely wound in the same manner as the round wire coil 11.

The metal round wire Wr used for the round wire coil 21 may or may not be the same as the metal round wire Wr used for the round wire coil 11. For example, the metal round wire Wr used for the round wire coil 21 may differ from the metal round wire Wr used for the round wire coil 11 in at least one of the diameter, the ratio B/A, the number of rows, and the material.

For example, the diameter of the round wire coil 21 may be greater than the diameter of the round wire coil 11.

In this case, the round wire coil 21 is hard to enter the gap S1 of the round wire coil 11, and it becomes easy to densely wind the round wire coil 21. Further, at the time of bending, the inner round wire coil 11 is easily bent, and the size of the gap S2 due to the bending can be suppressed. For this reason, the round wire coil 21 easily moves outward relative to each other along the outer peripheral portion of the round wire coil 11 at the time of bending, thereby the resistance to bending is reduced.

The flat wire coil 12 in the present embodiment is densely wound around the outer peripheral portion of the round wire coil 21 in the winding direction opposite to that of the round wire coil 21. For example, when the round wire coil 21 is S winding, the flat wire coil 12 is Z-winding.

The width, thickness, aspect ratio, number of rows, and material of the metal flat wire Wf used for the flat wire coil 12 can be determined in the same manner as in the first embodiment, depending on the diameter, ratio B/A, number of rows, and material of the metal round wire Wr used for the round wire coil 21.

The coil sheath 20 is a multi-layer coil that has a first inner layer Li1 (inner layer) formed by the round wire coil 11, a second inner layer Li1 (inner layer) formed by the round wire coil 21, and an outer layer Lo formed by the flat wire coil 12.

The coil sheath 20 is manufactured in the same manner as in the first embodiment except that the round wire coils 11 and 21 are formed into two layers in this order in the first process.

In the coil sheath 20 of the present embodiment, because the round wire coils 11 and 21 are disposed on the inner layer of the multi-layer coil and the flat wire coil 12 is disposed on the outer layer as in the first embodiment, rotational transmissibility is excellent even with a small outer diameter similarly to the first embodiment.

In particular, in the coil sheath 20 of the present embodiment, the inner layer of the multi-layer coil is made up of two layers of the round wire coils 11 and 21 having different winding directions. Since the round wire coils 11 and 21 are in contact with each other in a point shape at an intersection of the respective metal round wires Wr, the resistance at the time of bending is reduced as compared with the contact state between the metal round wire and the metal flat wire. As a result of reducing the bending resistance in the inner layer in this way, the resistance at the time of bending as the multi-layer coil as a whole is also reduced.

In each of the above embodiments, an example in which the inner layer made up of the metal round wires is one layer or two layers in the multi-layer coil of the medical coil has been described. However, the inner layer may be one or more layers, and is not limited to one or two layers. For example, the inner layer may be three or more layers.

The outer layer made up of the metal flat wires is not also limited to one layer. The outer layers may be two or more layers.

The inner layer in the multi-layer coil may be made up of one or more metal round wires. The outer layer of the multi-layer coil may be made up of one or more metal flat wires.

When at least one of the inner layer and the outer layer includes two or more layers, the inner layer that does not form the innermost layer is disposed radially inside the outer layer that does not form the outermost layer. That is, in the multi-layer coil, the winding layer made of the metal round wires is disposed radially inside the winding layer made of the metal flat wires.

The medical device may be an endoscopic medical device. For example, the medical device may be used by inserting through a treatment tool channel of an endoscope.

### EXAMPLE

Next, an example of the medical coil of each of the above-described embodiments will be described together with a modified example.

Following [Table 1] shows the configurations of Examples 1 to 3 and Modified example 1.

TABLE 1

| | | | | | Modified |
|---|---|---|---|---|---|
| | | Example 1 | Example 2 | Example 3 | Example 1 |
| Core metal | Outer diameter (mm) | ϕ1.8 | ϕ1.8 | ϕ1.8 | ϕ1.8 |
| Inner layer 1 | Material | SUS304WPB | SUS304WPB | SUS304WPB | SUS304WPB |
| | Type of wire | Round wire | Round wire | Round wire | Flat wire |
| | Size (mm) | ϕ0.18 | ϕ0.18 | ϕ0.18 | 0.45 × 0.18 |
| | Winding direction | S winding | S winding | Z winding | Z winding |
| | Number of row | 8 | 8 | 8 | 2 |
| Inner layer 2 | Material | — | — | SUS304WPB | — |
| | Type of wire | — | — | Round wire | — |
| | Size (mm) | — | — | ϕ0.18 | — |
| | Winding direction | — | — | S winding | — |
| | Number of row | — | — | 8 | — |
| Outer layer | Material | SUS304WPB | SUS304WPB | SUS304WPB | SUS304WPB |
| | Type of wire | Flat wire | Flat wire | Flat wire | Round wire |
| | Size (mm) | 0.45 × 0.18 | 0.36 × 0.18 | 0.36 × 0.18 | ϕ0.18 |
| | Winding direction | Z winding | Z winding | Z winding | S winding |
| | Number of row | 2 | 2 | 2 | 8 |

Example 1 is an example corresponding to the coil sheath 10 of the first embodiment.

As shown in [Table 1], the coil sheath 10 of Example 1 is a two-layer multi-layer coil having an inner layer 1 and an outer layer corresponding to the round wire coil 11 and the flat wire coil 12.

As the material of the inner layer 1, a metal round wire Wr made of SUS304WPB was used. The diameter of the metal round wire Wr was ϕ0.18 mm.

As the material of the outer layer, a metal flat wire Wf made of SUS304WPB was used. The cross-sectional shape of the metal flat wire Wf was a rectangular shape with rounded four corners, a width was 0.45 mm, and a thickness was 0.18 mm.

The coil sheath 10 of this embodiment was manufactured, using the method for manufacturing a medical coil of the first embodiment.

In the first process, the round wire coil 11 was formed by densely winding eight metal round wires Wr around the copper core metal 15 having a diameter of ϕ1.8. The winding direction was S winding. Both end portions of the round wire coil 11 were fixed by crimp terminals 13.

In the second process, the flat wire coil 12 was formed by densely winding two metal flat wires Wr around the outer peripheral portion of the round wire coil 11. The winding direction was Z winding. Both end portions of the flat wire coil 12 were fixed by crimp terminals 14.

After that, the multi-layer coil was introduced into a horizontal heat treatment furnace H-004 CSBCX (trade name; manufactured by Fuji Kagaku Kikai) to soften the core metal 15, and was subjected to heat treatment at 350° C. for 60 minutes.

After the core metal 15 was removed, the round wire coil 11 and the flat wire coil 12 were cut to a length of 2000 mm. Both end portions of the cut round wire coil 11 and flat wire coil 12 were brazed. As a result, a test sample S of the coil sheath 10 of Example 1 was manufactured.

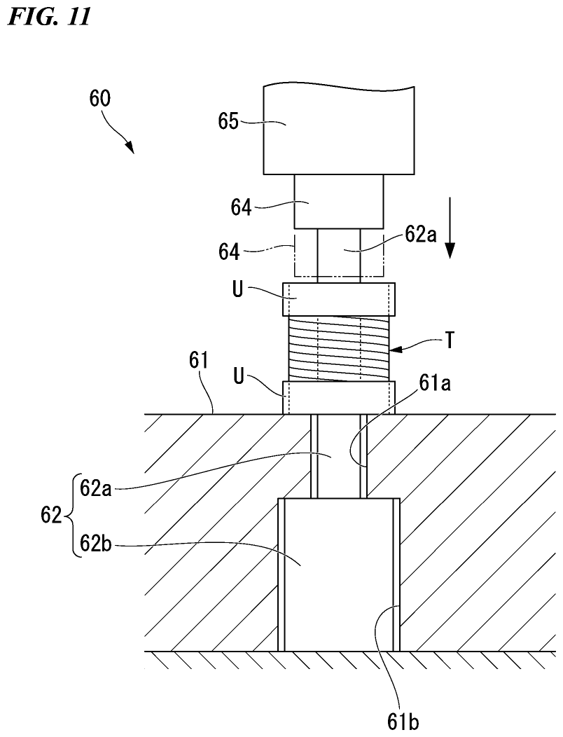

A test sample T similar to the test sample S was manufactured except that the length was 20 mm and both end portions were fixed by crimp terminals U (see FIG. 11).

### Example 2

Example 2 is an example corresponding to the coil sheath 10 of the first embodiment.

As shown in Table 1, the coil sheath 10 of the second embodiment is the same as the coil sheath 10 of the first embodiment, except that a width of the metal flat wire Wt forming the flat wire coil 12 of the outer layer is 0.36 mm.

The test samples S and T were also manufactured in the coil sheath 10 of this example.

### Example 3

Example 3 is an example corresponding to the coil sheath 20 of the second embodiment.

As shown in Table 1, the coil sheath 20 of Example 3 was a three-layer multi-layer coil having an inner layer 1, an inner layer 2, and an outer layer corresponding to the round wire coils 11 and 21, and the flat wire coil 12.

The inner layer 1 is the same as the inner layer 1 of the first embodiment, except that it is formed by being wound around a core metal having a diameter of ϕ1.4 mm and the winding direction is Z winding.

The inner layer 2 was formed in the same manner as the inner layer 1, except that two metal round wires Wr similar to the inner layer 1 were densely wound around the outer peripheral portion of the round wire coil 11 by S winding.

The outer layer was formed in the same manner as the outer layer of Example 1, except that the outer layer was densely wound around the outer peripheral portion of the round wire coil 11 by S winding.

The test samples S and T were also manufactured in the coil sheath 20 of this example.

### Modified Example 1

Modified example 1 is the same as Example 1, except that the outer layer of Example 1 is disposed on the inner layer and the inner layer of Example 1 is disposed on the outer layer.

The test samples S and T were also manufactured in the coil sheath of this modified example.

### Evaluation

As shown in Table 2 below, the rotational transmissibility and compressive resistance were evaluated, using the test samples S and T of the coil sheaths of Examples 1 to 3 and Modified example 1.

TABLE 2

| | Rotational transmissivity | | Compressive resistance | | |
|---|---|---|---|---|---|
| | Tip rotation angle | | Spring constant | | Comprehensive |
| | (deg) | evaluation | (kN/mm) | evaluation | evaluation |
| Example 1 | 8.94 | A | 1.69 | A | A |
| Example 2 | 13.94 | A+ | 1.69 | A | A |
| Example 3 | 30.58 | A+ | 2.28 | A | A |
| Modified | 0.00 | B | 0.85 | B | B |
| example 1 | | | | | |

### [Rotational Transmissibility]

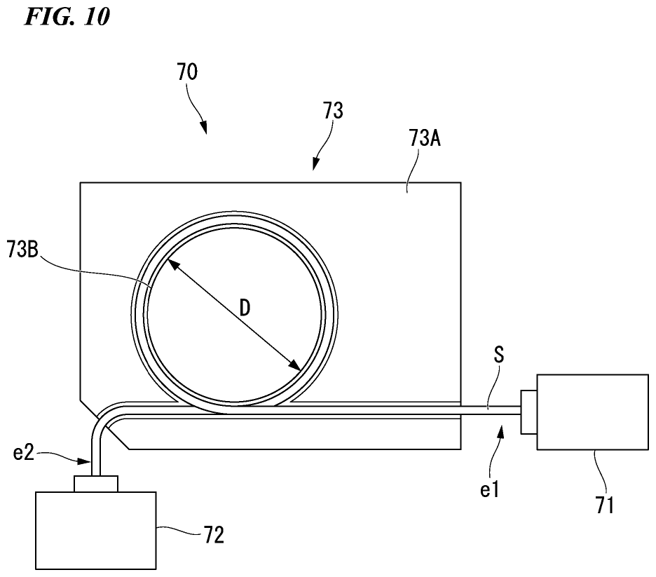

FIG. 10 is a schematic diagram showing a test device of rotational transmissibility.

As shown in FIG. 10, a test device 70 has a sheath rotation portion 71, a rotation angle detection unit 72, and a sheath holder 73.

The sheath rotation portion 71 grasps a first end portion el of the test sample S and rotates the test sample S by a certain angle in the circumferential direction.

The rotation angle detection unit 72 detects the rotation angle of a second end portion e2 on the opposite side of the first end portion e1 in the test sample S. An angle detection sensor was used for the rotation angle detection unit 72.

The sheath holder 73 keeps the bent shape of the test sample S constant while the test sample S is rotated. The sheath holder 73 includes a flat plate-shaped base 73A and a guide portion 73B formed on the base 73A. The guide portion 73B is formed by a U-shaped groove in which the test sample S linearly extending from the sheath rotation portion 71 is circulated along a circle having a diameter D, rotated by 360 degrees, and further bent 90 degrees. The size of D was set to 200 mm.

The rotational transmissibility was expressed by the rotation angle of the second end portion e2 when the test sample S is rotated by 45 degrees at the first end portion e1 (described as “tip rotation angle” in Table 2). The unit of the tip rotation angle was degrees (described as “deg” in Table 2).

The rotational transmissibility is better as it is closer to 45 degrees. If the tip rotation angle is 0 degrees or more and less than 5 degrees, it was evaluated as bad (not good, “B” in Table 2). If the tip rotation angle is 5 degrees or more and less than 10 degrees, it was evaluated as good (good, “A” in Table 2). If the tip rotation angle is 10 degrees or more and 45 degrees or less, it was evaluated as very good (very good, “A+” in Table 2).

### [Compressive Resistance]

FIG. 11 is a schematic diagram showing an example of the compressive resistance test device.

As shown in FIG. 11, the compressive resistance of the coil sheath of Examples 1 to 3 and Modified example 1 was evaluated using a test sample T having a total length of 20 mm in which both end portions were fixed by crimp terminals U. Each crimp terminal U was provided within a range of 5 mm from the end face of the test sample T.

As the measured value of compressive resistance, the spring constant at the time of compression in the axial direction of the test sample T was used.

The test device 60 has a pedestal 61, a guide pin 62, a pressing head 64, and a load cell 65. The test device 60 further includes a control unit and a calculation device of the measured value which are not shown.

The pedestal 61 is a disc-shaped member having high rigidity. In the thickness direction of the pedestal 61, a through hole 61a and a hole portion 61b are formed from the upper side to the lower side. An inner diameter of the through hole 61a is smaller than an inner diameter of the test sample T. The inner diameter of the hole portion 61b is larger than that of the through hole 61a.

The guide pin 62 has a rod 62a and a pressure plate 62b.

The rod 62a has an outer diameter that can be inserted through the through hole 61a and the inside of the test sample T. The rod 62a is inserted into the through hole 61a from below and protrudes above the pedestal 61.

The pressure plate 62b has an outer shape that can be inserted into the hole portion 61b and cannot be inserted into the through hole 61a, and is fixed to a lower end portion of the rod 62a.

By pressing the upper end portion downward, the rod 62a can move on the pedestal 61 from a state in which the rod 62a protrudes longer than the total length of the test sample T to a state in which the rod 62a protrudes shorter than the total length of the test sample T.

The pressing head 64 is connected to an elevating device (not shown) via the load cell 65. The pressing head 64 is disposed to be movable up and down above the rod 62a.

The pressing head 64 presses the upper end of the test sample T inserted through the rod 62a from above toward the pedestal 61. The pressing head 64 is provided with a hole into which the tip portion of the rod 62a is inserted. Therefore, when pressing the test sample T, the pressing load is not transferred to the rod 62a.

The load cell 65 measures the load acting on the pressing head 64.

The spring constant of the test sample T was measured by the test device 60 as follows.

The test sample T was inserted into the rod 62a protruding from the pedestal 61, and the test sample T was placed on the pedestal 61.

After that, as shown by an alternate long and short dash line, the pressing head 64 descended, and the test sample T disposed on the pedestal 61 was compressed in the axial direction. The magnitude of the load to be output from the load cell 65 was acquired at each descending position of the pressing head 64. The test device 60 calculated the spring constant of the test sample T at the time of compression from a relationship between the descending position and the magnitude of the load.

The unit of the spring constant was KN/mm.

### [Evaluation Results]

As shown in Table 2, the measured values of the tip rotation angles of Examples 1 to 3 and Modified example 1 in the rotational transmissibility test are 8.94 degrees, 13.94 degrees, 30.58 degrees, and 0.00 degrees, respectively. The rotational transmissibility was determined to be good (A) in Example 1, very good (A+) in Examples 2 and 3, and bad (B) in Modified example 1. However, since the spring constant of Example 3 was about 2.2 times that of Example 2, Example 3 was significantly superior to Example 2 in rotational transmissibility.

It is considered that the reason why the rotational transmissibility of Example 2 is superior to that of Example 1 is because the width w of the flat wire coil 12 of Example 1 is 2.5 times the diameter d of the round wire coil 11, on the other hand, w is considered to be twice d in Example 2.

In the rotational transmissibility test, a loop in which the test sample S rotates by 360 degrees is drawn. Therefore, when the proximal end side is rotated, the bending amount and bending direction with respect to the test sample S change in the loop portion as the rotation amount increases. Therefore, as the test sample S can be bent with less resistance, the rotational transmissibility is better.

It is considered that the rotational transmissibility is better because the flat wire coil 12 is easily bent when the width w of the flat wire coil 12 is twice or less the diameter d of the round wire coil 11.

It is considered that the reason why a rotational transmissibility in Example 3 is superior in to Examples 1 and 2 is because w/d is doubled and the inner layer is a two-layer structure of round wire coils 11 and 21 in the Example 3. Since the round wire coils 11 and 21 having different winding directions are in point contact with each other, the round wire coils 11 and 21 smoothly slide relative to each other at the time of bending. This reduces the resistance at the time of bending.

In the case of Modified example 1, since the tip rotation angle was 0.00 degrees, the tip could not be rotated only by turning 45 degrees at the proximal end. The reason for this is because the round wire coil 11 enters the gap of the flat wire coil 12 on the outside of the bend at the time of bending, and the resistance when bending in the opposite direction becomes too large.

As shown in Table 2, the measured values of the spring constants of Examples 1 to 3 and Modified example 1 in the compressive resistance test were 1.69 kN/mm and 1.69 kN/mm, 2.28 kN/mm and 0.85 kN/mm, respectively. The compressive resistance was determined to be good (A) in Examples 1 to 3 and bad (B) in Modified example 1.

It is considered that the reason which the spring constants of Examples 1 and 2 were the same is because the cross-sectional area and the material were the same and the dense winding property was also the same.

In contrast, Modified example 1 is the same as Examples 1 and 2 in terms of cross-sectional area and material. Accordingly, it is considered that the reason why the spring constant was low is because the dense winding property was inferior. That is, when the round wire coil 11 enters the gap on the flat wire coil 12, the winding directions are not uniform, and a gap is generated between the windings adjacent to each other. Since the lowering amount of the pressing head 64 includes the lowering amount for eliminating the gap between the windings, the spring constant deteriorates.

It is considered that the reason why the spring constant of Example 3 was larger than that of Examples 1 and 2 is because the cross-sectional area of the test sample T was larger in Example 3.

Although the respective preferred embodiments and respective examples of the present invention have been described above, the present invention is not limited to such embodiments and examples. It is possible to add, omit, replace, and make other changes to the configuration without departing from the spirit of the present invention. Further, the present invention is not limited by the above description, but is limited only by the claims of attachment.

What is claimed is:

1. A medical coil comprising a multi-layer coil including a plurality of winding layers in which metal strands are spirally wound, wherein the multi-layer coil comprising:
an inner layer made up of one or more metal round wires; and
an outer layer made up of one or more metal flat wires, wherein
the inner layer and the outer layer are contact with each other.

2. The medical coil according to claim 1, wherein a width of the metal flat wires is 1 times or more and 2 times or less a diameter of the metal round wires.

3. The medical coil according to claim 1, wherein the multi-layer coil includes two of the inner layers disposed at different positions in a radial direction of the medical coil, and wherein
the outer layer disposed outside the two inner layer.

4. The medical coil according to claim 1, wherein the metal round wires and the metal flat wires are densely wound.

5. A method for manufacturing a medical coil, the method comprising:
a first process of forming at least one layer of round wire coil by winding one or more metal round wires around a core metal; and
a second process of forming a flat wire coil by winding one or more metal flat wires around an outermost periphery of the round wire coil such that the flat wire coil is contact with the outermost periphery of the round wire coil.

6. The method for manufacturing the medical coil according to claim 5, wherein a width of the metal flat wires is 1 times or more and less than 2 times an outer diameter of the metal round wires.

7. The method for manufacturing the medical coil according to claim 5, wherein the round wire coil is formed in two layers in the first process.

8. The method for manufacturing the medical coil according to claim 5, wherein the metal round wires and the metal flat wires are densely wound in the first process and the second process.

9. A medical device comprising the medical coil according to claim 1.
